(19) United States
(12) Patent Application Publication
(10) Pub. No.: US 2011/0166510 A1
(43) Pub. Date: Jul. 7, 2011

(54) Portable IV infusion monitoring system

(76) Inventors: Jun Feng, Lincoln, MA (US); Xueming Miao, Wuxi (CN)

(21) Appl. No.: 12/932,990

(22) Filed: Mar. 11, 2011

Related U.S. Application Data
(63) Continuation-in-part of application No. 11/811,466, filed on Jun. 10, 2007.
(60) Provisional application No. 60/814,238, filed on Jun. 16, 2006.
(60) Provisional application No. 60/815,204, filed on Jun. 20, 2006.

(51) Int. Cl. A61M 5/168

(52) U.S. Cl. 604/65; 604/253

(57) ABSTRACT

A portable infusion monitoring system detects the liquid level and flow rate during infusion process, as well as gives an alarm for patents and nurses in hospital as the medical liquid in an IV bottle drops to a predetermined low level. This system comprises a set of liquid level sensor, a microprocessor, and a monitor terminal. The liquid level sensor generates an electric signal related to the liquid level inside the IV bottle. The microprocessor statistically analyzes the electric parameters detected from the electric signal, and obtains the liquid level data including both the liquid level and liquid flow rate. The liquid level data are sent to the monitor terminal for display, and an alarm is activated when the medical liquid inside the IV bottle drops to a predetermined low lever. Further functions of the monitor terminal includes an automatic switch to cut off the IV feeding process and send the alarm signal to a nurse station through signal network by wire or wirelessly. Several interference filtering methods are applied to increase signal/noise ratio, and therefore warrant the operation reliability.

### CROSS REFERENCE TO RELATED APPLICATIONS

This application is a CIP of Ser. No. 11/811,466 filed on Jun. 10, 2007 and RCE filed on Feb. 16, 2010, by the present inventors to US Patent and Trademark Office. It is also noted that the above mentioned non-provisional applications claim the benefit of PPA Ser. No. 60/814,238, filed on Jun. 16, 2006, and Ser. No. 60/815,204 filed on Jun. 20, 2006, by the present inventors to US Patent and Trademark Office.

### REFERENCES CITED

U.S. PATENT DOCUMENTS

| | | | |
|---|---|---|---|
| 3,375,716 | Apr. 2, 1968 | Hersch | 73/304 |
| 3,390,577 | Jul. 2, 1968 | Phelps et al. | 73/194 |
| 3,450,153 | Jul. 17, 1969 | Hildebrandt et al. | 137/486 |
| 3,641,543 | Feb. 8, 1972 | Rigby | 73/861.41 |
| 3,939,360 | Feb. 17, 1976 | Jackson | 307/118 |
| 4,002,996 | Jan. 11, 1977 | Klebanoff et al. | 331/65 |
| 4,470,008 | Sep. 4, 1984 | Kato et al. | 324/61 |
| 4,671,110 | Jun. 9, 1987 | De Kock | 73/323 |
| 4,749,988 | Jun. 7, 1988 | Berman et al. | 340/618 |
| 5,563,584 | Oct. 8, 1996 | Rader et al. | 340/618 |
| 6,964,278 | Nov. 15, 2005 | Tschanz | 137/392 |
| 11/140,087 | Feb. 2, 2006 | Cassidy | 604/253 |

### FEDERALLY SPONSORED RESEARCH

Not Applicable

### SEQUENCE LIST OR PROGRAM

Not Applicable

### FIELD OF INVENTION

The present invention relates to a portable monitoring system of the liquid level in a medical liquid feeding line, and more particularly to a portable IV infusion monitoring system.

### BACKGROUND OF THE INVENTION

Assume a patient lies on bed to receive IV infusion. There are two types of infusion systems. One is by pump, another is by gravity. The pump infusion system is very costly and often encounters maintenance trouble. Therefore, many hospital workers prefer to use the traditional gravity infusion system. The gravity IV infusion line consists of three parts: a) An IV bottle contains medical liquid and air above the medical liquid; (b) Infusion line includes a liquid needle inserted inside the IV bottle to receive medical liquid, a plastic tube (liquid tube) with one end connected to the liquid needle as liquid inlet and another end connected to the IV injection needle for injecting the medical liquid into the patient vein. A flow rate switch is located in the middle of the plastic tube to control the flow rate manually; (c) Air line includes an air needle inserted into the IV bottle to apply air pressure for driving the liquid flow, and a plastic tube (air tube) with one end connected to the air tube as air outlet and another end opened to the environment as air inlet. As the medical liquid in the IV bottle drops to a predetermined low level, i.e., nearly finished, the bottle must be replaced by a new one, otherwise air may enter the infusion line and causes serious medical problems.

So far, the job of bottle replacement needs frequent supervision from patient and nurses by eyeball. This task becomes a heavy burden of medical workers, particularly at night. To develop a monitoring system for IV infusion becomes a big demand from hospitals and patients. Many efforts have been done in this field.

U.S. Pat. No. 3,375,716 to Hersch discloses a fluid quantity measuring device including a sensing capacitor to measure the prevailing quantity of fluid in a container. Hersch's disclosure uses a time-constant circuit, and therefore the measurement accuracy is very poor as well the poor reliability, both of which are very critical in medical application. The present invention applies a microprocessor including an interference filtering means, which acts as a mini computer to process all received electric parameters from the electrodes in digital format, and therefore warrant a very high accuracy and high reliability. Further more, the present invention uses sound alarm, terminal display and signal network to further ease the hospital works.

U.S. Pat. No. 3,390,577 to Phelps et al. discloses a monitoring system for fluid flow in drop form. Phelps' disclosure only applies for measuring the liquid drop. Such system is poor in accuracy and reliability. The present invention can measure the liquid level data at any time moment. Further more, the present invention applies a microprocessor, which acts as a mini computer to process all received electric parameters from the electrodes in digital format, and therefore warrant a very high accuracy and high reliability. In addition, the present invention uses sound alarm, terminal display and signal network to further ease the hospital works.

U.S. Pat. No. 3,641,543 to Rigby discloses a low-level detector and drop rate monitor that can only detect the low solution level and drop rate. Rigby's first embodiment is for detecting a low solution level, where the conductor means needs to be mounted on opposite one another in juxtaposition, one of the conductors and a multivibrator means are required to be grounded. His second embodiment is for monitoring drop rate, where the two electrode means must be placed diametrically opposite one another in juxtaposition, a stabilizing means and a tachometer means are required. The present invention can detect all liquid infusion information including the liquid level at any time moment and liquid moving rate including the low liquid level. Furthermore the present invention does not require two electrodes being placed on opposite one another, does not require any element to be grounded and therefore is portable. In addition, the present invention does not need stabilizing means and tachometer means for operation.

U.S. Pat. No. 3,939,360 to Jackson discloses a liquid level sensor and electrode assembly therefore. Jackson's disclosure requires three capacitance plates to measure the capacitance. Furthermore, the circuit means uses analog signal for measurement, and therefore results in a poor accuracy and poor reliability. The present invention needs only minimum two electrodes for measurement, and the signal process is accomplished by a microprocessor, therefore, warrant a high accuracy and high reliability.

U.S. Pat. No. 4,002,996 to Klebanoff et al. discloses a level detector using oscillator circuit with two capacitive probes. Klebanoff's disclosure detects the low liquid level by emitting an oscillation using a feed-back network. The present invention applies the received signal to microprocessor and send out an alarm signal when the microprocessor analyzes the digital data and finds that the liquid level has dropped to a predetermined low level.

U.S. Pat. No. 4,470,008 to R. Kato et al. discloses a capacitance sensor for detecting liquid level. Kato's disclosure requires three electrodes to measure the capacitance. Furthermore, it applies an analog circuit for measurement, and therefore results in a poor accuracy and poor reliability. The present invention needs only minimum two electrodes for measurement, and the signal process is accomplished by a microprocessor, therefore, warrant a high accuracy and high reliability.

U.S. Pat. No. 4,671,110 to de Kock discloses a level sensing device. De Kock's disclosure is for sensing the liquid level in a boiler or vessel, and therefore need to have tubular glass and a conduit for communication with the liquid inside the vessel. One of the conductors needs to contact the liquid inside the vessel. The present invention is for detecting the liquid level in an IV bottle, and does not need any contact with the liquid inside the liquid container.

U.S. Pat. No. 4,749,988 to Berman et al. discloses a non-invasive liquid level sensor. Berman's disclosure requires the outer shield conductor of a shielded cable to be grounded in order to avoid external interference to the electrode, and therefore such a sensor is not portable. Furthermore, his disclosure does not include any signal process element and signal terminal equipment. The present patent does not need any part to be grounded and therefore is portable. The present invention includes a microprocessor acting as a mini computer, and all the interferences from environment are processed in the microprocessor to be filtered out. Furthermore, the present invention includes the monitor terminal for alarm and display.

U.S. Pat. No. 5,563,584 to Rader et al. discloses a liquid level sensing and monitoring system for medical fluid infusion systems. Rader's disclosure applies pressure sensor technology. In his second embodiment, a sensor is inserted into the outlet of a liquid container and contacts the liquid for detecting the liquid level. The present invention applied the impedance sensor, and none of elements in the present invention needs to be inserted into the outlet of a liquid container.

U.S. Pat. No. 6,964,278 to Tschanz discloses a non-invasive gauge glass liquid level sensor apparatus. Tschanz's disclosure is for sensing liquid level in a boiler or other vessel. Therefore his apparatus requires a tubular gauge glass. In addition, the boiler or vessel must be metallic material. The present invention is for monitoring IV infusion liquid level, and does not requires a gauge glass as well as a metallic material for the liquid container.

U.S. patent application Ser. No. 11/140,087 to Cassidy discloses 3 different embodiments: detecting air bubbles trapped inside liquid, an active gas removal system and an IV flow control system, none of any Cassidy's disclosures is related to an IV monitoring system of the present invention. Furthermore, Cassidy's system needs 3 or 4 electrodes for sensing, but the present invention only needs 2 electrodes.

The present invention provides a portable IV infusion monitoring system, which only needs minimum 2 electrodes for a liquid level sensor and is capable for detecting and displaying both the liquid level and liquid flow rate, as well as giving alarm when the medical liquid in the bottle drops to a predetermined low level. The present invention is different from and superior over all the prior arts in structure, cost, accuracy and reliability, as well as in ease of use.

### SUMMARY OF THE INVENTION

A portable IV infusion monitoring system is provided to monitor the liquid level and to give alarm as the medical liquid in the IV bottle drops to a predetermined low level. The IV infusion is used for injecting a medical liquid to a patient vein. It includes an IV bottle containing medical liquid in lower part and air above the medical liquid. The IV bottle, as the supplier of the IV infusion medical liquid, comprises at least one of a glass bottle, a plastic bottle, and a plastic bag. The liquid level is defined as the interface between the medical liquid and the air above the medical liquid in the IV bottle. Both a liquid needle for liquid flow and an air needle for air flow are inserted into the IV bottle. A plastic liquid tube for liquid flow is connected at the end of the liquid needle. A plastic air tube for air flow is connected at the end of the air needle.

The preferred embodiment of the present invention comprises a set of liquid level sensor including at least two electrodes, a microprocessor, and a monitor terminal. The power is provided preferably by a battery or an external power source as an option to user. The at least two electrodes are located at either two sides of the IV bottle in opposite direction or one side of the IV bottle in parallel location. They are capable of conducting an electric current between them, e.g., an alternating current. The microprocessor acting as a mini computer is capable of detecting the electric parameters of the alternating current, converting the detected electric parameters into a group of digital data points, analyzing the group of digital data points by statistics to obtain the liquid level data inside the IV bottle, and sending all the liquid level data to the monitor terminal. The electric parameters related to the liquid level include at least one of voltage, current, impedance, phase and frequency etc. The liquid level data include the liquid level inside the IV bottle at any time moment, the liquid flow rate during infusion process, and the comparison with the predetermined low level. The monitor terminal includes an alarm means for sending an alarm, and a display means to display the liquid level data in a terminal screen. Alternatively, the liquid level sensor uses an electric bridge to detect the electric signal for better accuracy.

The microprocessor acting as a mini computer includes a control means for applying the alternating current to the at least two electrodes, receiver means for receiving the electric signal of the alternating current, detector means for detecting the electric parameters of the electric signal, converter means for converting the detected analog electric parameters into a group of digital data, process means for analyzing the group of digital data by statistics, which results in the liquid level data inside the IV bottle, as well as transmission means for sending out the liquid level data. Each of above elements may be built together in one chip, or they can stand alone as individual circuit or chip. The control means includes at least one of an oscillator, an oscillator circuit, a logic circuit etc. The receiver means includes at least one of an input pot, an amplifier, a filter etc. The detector means includes at least one of a CN converter (capacitance to voltage converter), a differential circuit, or a voltage meter etc. The converter means includes at least one A/D converter. The process means includes at least one of signal interface, digital register, processor, or logic circuit etc. The transmission means includes at least one of output pot, conductive wire or antenna etc.

Alternatively the microprocessor includes receiver for receiving the electric signal of the alternating current, detector for detecting the voltage signal from the electric signal, signal interface for storing the voltage signal, A/D converter for converting the voltage signal into a group of digital data points, digital register for storing the digital data, processor for analyzing the group of digital data points by statistics and obtaining the liquid level data simultaneously, output port for transmitting the liquid level data. All the functions of each element are controlled by program controller, which is programmed with unique software code for administrating the operation of all above elements. Each of above elements may be built together in one chip, or they can stand alone as individual circuit or chip. The microprocessor further includes an interference filtering means for removing all interference from the environment.

The electric interference from environment often degrades or sometime disables the normal operation of such a monitoring system. Therefore, to move the signal interference becomes very critical in order to obtain high accuracy and high reliability of the monitoring work. In a typical electric environment, at least one shielding plate made of conductive materials is placed on the outer surface of each electrode. The shielding plate is insulated to the electrodes. The at least one shielding plate is connected to a reference point with zero potential, e.g., the negative pole of a battery. Alternatively, the interference noise in the at least one shielding plate is passed over to the microprocessor, and it is then filtered out in signal processing. Meanwhile, at least two coaxial cables consist of a center conductor surrounded by a concentric outer shielding layer made of conductive materials. The center conductor is insulated from the outer shielding layer. The center conductors of the at least two coaxial cables connect the at least two electrodes to the microprocessor for transmitting the signal. The outer shielding layers of the at least two coaxial cables are connected to the reference point with zero potential, e.g., the negative pole of a battery. Alternatively, the outer shielding layers are connected to the microprocessor for interference filtering process. The microprocessor, part of the monitor terminal and the battery are contained in an assembly box. To shielding the microprocessor and other parts inside the assembly box from the environmental interference, either the assembly box is made of metal or the assembly box is coated with conductive materials. The coated methods include chemical coating, physical coating, mechanical coating, or a simple metal lining. Similar to the shielding plate, the conductive part of the assembly box is connected to a reference point with zero potential, e.g., the negative pole of the battery. Alternatively, the environmental noise in the assembly box is passed over to the microprocessor for interference filtering.

However, if the electric environment is very noisy, and the interference becomes too strong to perform a normal operation of this monitoring system, the signal interference from the environment can be removed by special signal processing methods. The control means in the microprocessor generates the alternating current in various forms such as narrow band signal, multi-frequency signal, and encoded signal (containing continuous wave, pulse and digital signal etc.). If a narrow band signal is applied, the interference filtering means in the microprocessor has a narrow band filter, which can filter out the signal within this narrow band, and remove all random interference outside the narrow band. If a multi-frequency signal is applied, the interference filtering means has a Fourier analyzer, which can perform Fourier analysis to pick up the right signal, and remove the noise interference. If an encode signal is applied, the interference filtering means has a decoder, which can perform decoding to pick up the right signal, and remove the noise interference. The way of encoding includes frequency modulation, angle modulation, phase modulation, pulse modulation, pulse code modulation, FDMA and CDMA modulations etc. All above filtering methods are more effective in digital format

Further alternatively, two pairs of electrodes can be positioned in parallel outside the IV bottle 11. By differentiation of the signal or electric parameters, the environmental interference will be removed too. Hereby there is no need of grounding in order to avoid the environmental interference since this monitoring system is designed as a portable device.

In addition to the environmental interference, the signal deformation may also reduce the reliability of the monitoring system, e.g., in the case of flexible IV bag (i.e., a soft IV bottle), the bag may deform during infusion process and therefore lead to the deformation of the electrical signal and related electrical parameters. However, such signal deformation can be analyzed by the microprocessor, and the corrected electric parameters can be picked up by the analysis. Therefore, it would be impossible to obtain high accuracy and high reliability without the microprocessor.

The monitor terminal includes alarm means for providing an alarm, and display means for displaying the liquid level data in a terminal screen. The alarm means includes a sound generator for giving a loud sound when the medical liquid level inside the IV bottle drops below the predetermined low level. Alternatively the alarm means includes a switch means for cutting off the feeding of medical liquid when the medical liquid level inside the IV bottle drops below the predetermined low level. Further alternatively, the alarm means includes a signal network for sending the liquid level data by the network to a nurse station through wire or wirelessly.

The monitor terminal further includes a rate controller for controlling the infusion rate according to a predetermined rate value. The rate controller comprises an input port for inputting the desired infusion rate of the medical liquid inside the IV bottle, a comparator for comparing the desired infusion rate and the detected infusion rate, and an electric switch means for adjusting the infusion rate according to the results from the comparator.

The preferred embodiment can be further simplified as the followings: A portable IV infusion monitoring system comprises

(a) A liquid level sensor consists of two electrodes. An IV bottle comprises at least one of a glass bottle, a plastic bottle, and a plastic bag, the IV bottle contains medical liquid in lower part and air above the medical liquid. The two electrodes are positioned outside the IV bottle for conducting an electric current between the two electrodes, and for detecting liquid level of the medical liquid. The liquid level is defined as the interface between the medical liquid and the air above the medical liquid in the IV bottle. Therefore, the statement of “detecting liquid level of the medical liquid” is equivalent to say “measuring the interface between the medical liquid and the air above the medical liquid in the IV bottle.” Each of the electrodes is made of only one piece of metal or other conductive material, and therefore the Kato's reference includes 3 electrodes to make up the transducers;

(b) A microprocessor includes control means for controlling the electric current between the two electrodes, receiver means for receiving an electric signal of the electric current, detector means for detecting electric parameters of the electric signal, the electric parameters comprising voltage or impedance, A/D converter means for converting the electric parameters into a group of digital data points, process means having a program or software for statistically analyzing the group of digital data points and obtaining both liquid level and liquid flow rate simultaneously by the statistical analysis, as well as transmission means for sending out the liquid level and liquid flow rate to a monitor terminal. The statistical analysis includes at least one of data filtering, curve fitting and statistical modeling to a group of numerous data points as a function of time, and statistical method to remove electronic noise due to environment. It must be indicated that the applied statistical analysis of the present invention comprises 2 key factors: (1) measuring a group of data points as a function of time; (2) using statistical technique, e.g. curve fitting and modeling etc. The results are obtained from the modeled curve. Hereby, the statistical analysis excludes any algebra method, e.g., comparing one point to another point in Cassidy's reference;

(c) The monitor terminal has alarm means responsive to the liquid level data for giving alarm;

(d) a battery for providing an electric power to the monitoring system, the battery has a negative pole for providing a zero potential reference point to the monitoring system, all parts in the monitoring system are non-grounded, the “portable” is defined as a function such that the IV infusion monitoring system can operate while moving around with a patient.

### BRIEF DESCRIPTION OF THE DRAWINGS

FIG. 1 is a schematic drawing of the preferred embodiment of a portable IV infusion monitoring system for the present invention.

FIG. 2 is the schematic drawing of an alternative embodiment of the present invention.

FIG. 3 is a schematic drawing of an exemplary liquid level sensor for the present invention.

FIG. 3A is a schematic drawing of an exemplary coaxial cable, shielding plate and assembly box for removing the environmental interference in the present invention.

FIG. 4 is a block diagram of an exemplary microprocessor for the present invention.

FIG. 4A is a block diagram of an alternative microprocessor for the present invention.

FIG. 4B is a block diagram of three alternative embodiments of the interference filtering means in the microprocessor for the present invention.

FIG. 5 is a block diagram of an exemplary monitor terminal for the present invention.

FIG. 5A-5C are block diagrams of exemplary and alternative alarm means for the present invention.

FIG. 5D is a block diagram of an alternative monitor terminal for the present invention.

FIG. 6 is a schematic diagram of an exemplary statistical analysis for the present invention.

### DETAILED DESCRIPTION OF THE ILLUSTRATIVE EMBODIMENTS

In describing preferred embodiment of the present invention illustrated in the drawings, specific terminology is employed for the sake of clarity. However, the invention is not intended to be limited to the specific terminology so selected, and it is to be understood that each specific element includes all technical equivalents which operate in a similar manner to accomplish a similar purpose.

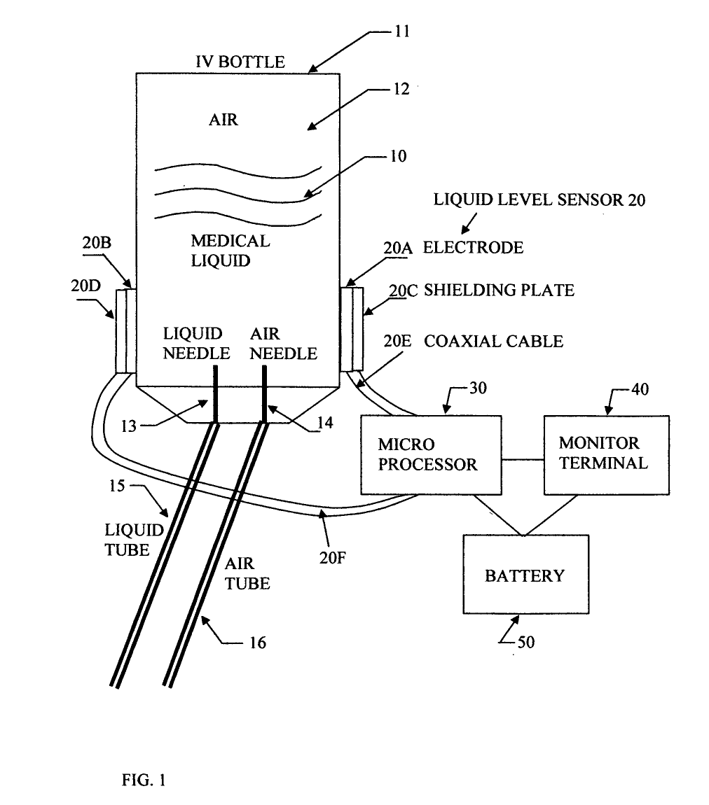

FIG. 1 is a schematic drawing of the preferred embodiment of a portable IV infusion monitoring system that is capable of detecting the liquid level of the medical liquid 10 inside an IV bottle 11, and giving alarm when the medical liquid 10 in the IV bottle 11 drops to a predetermined low level.

The IV infusion system comprises the IV bottle 11 containing the medical liquid 10 in the lower part and air 12 above the medical liquid 10. The IV bottle 11 being the supplier of the IV infusion medical liquid 10 comprises at least one of a glass bottle, a plastic bottle, and a plastic bag. The liquid level is defined as the interface between the medical liquid 10 and the air 12 above the medical liquid 10 in the IV bottle 11. Therefore, the statement of “detecting liquid level of the medical liquid 10” is equivalent to say “measuring the interface between the medical liquid 10 and the air 12 above the medical liquid 10 in the IV bottle 11.” A liquid needle 13 and an air needle 14 are inserted into the IV bottle 11. A liquid tube 15 is connected at the end of the liquid needle 13. An air tube 16 is connected at the end of the air needle 14. The IV bottle 11 can be made of stiff materials such as glass or harden plastics, or it can be made of flexible plastic bags.

The IV infusion monitoring system comprises a liquid level sensor 20 including at least two electrodes 20A, 20B, a microprocessor 30, and a monitor terminal 40. The power is provided preferably by a battery 50 or by an external power source as an option to user. The at least two electrodes 20A, 20B are located at two sides of the IV bottle 11 in opposite direction with each other, and are capable of conducting an alternating current between them. Each electrode 20A, 20B is made of one piece of metal or other conductive material, and it must be insulated with other conductive material in a sensing system. The microprocessor 30 acting as a mini computer is capable of detecting the electric parameters of the alternating current, converting the detected electric parameters into a group of digital data points, analyzing the group of digital data points statistically to obtain the liquid level data inside the IV bottle 11, and sending all the liquid level data to the monitor terminal 40. The electric parameters related to the liquid level include at least one of voltage, current, impedance, phase and frequency etc. The liquid level data includes the liquid level inside the IV bottle 11 at any time moment, the liquid flow rate during infusion process, and the comparison with the predetermined low level. The monitor terminal 40 includes an alarm means for sending out an alarm signal to activate an alarm to patient and nurses if the medical liquid 10 has dropped to the predetermined low level.

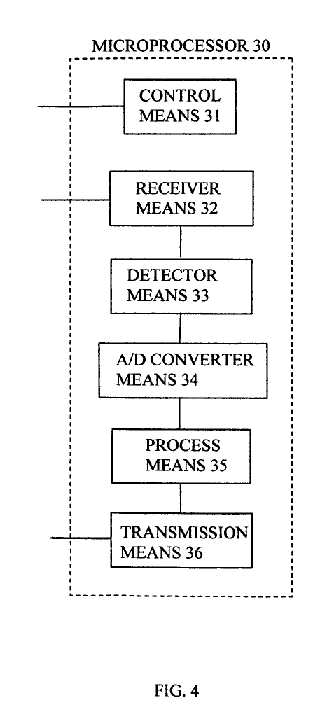

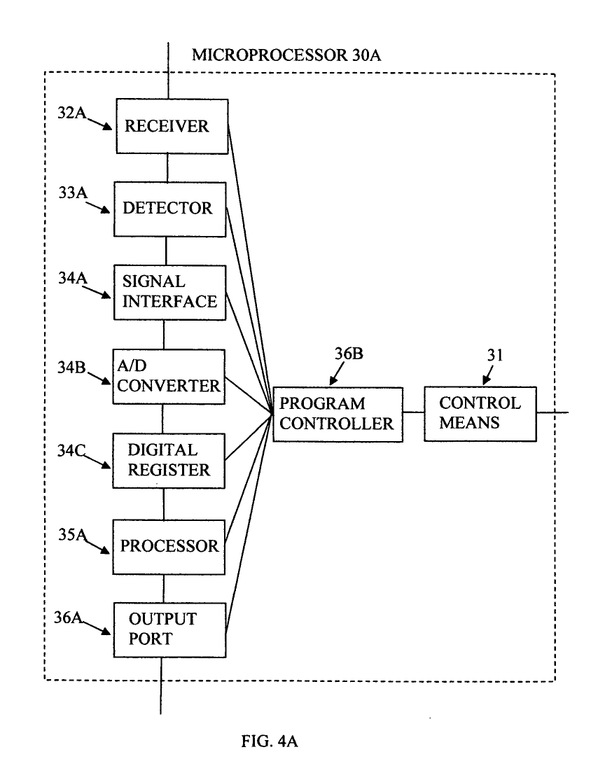

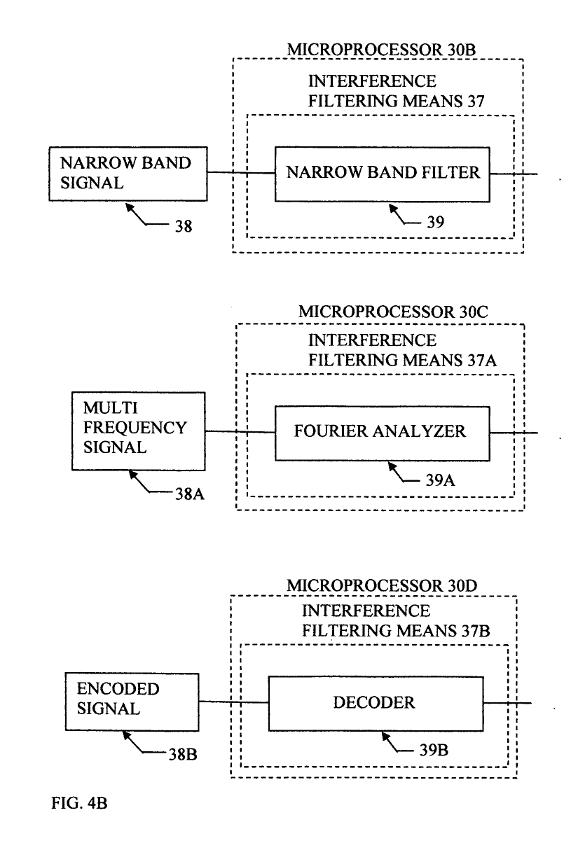

In a typical electric environment, at least one shielding plate 20C, 20D made of conductive materials is placed on the outer surface of each electrode 20A, 20B, and is insulated from the electrodes 20A, 20B. The at least one shielding plate 20C, 20D is connected to a reference point with zero potential, e.g., the negative pole of a battery 50. Alternatively, the interference noise in the at least one shielding plate is passed over to the microprocessor 30, and it is then filtered out in signal processing. Meanwhile, at least two coaxial cables 20E, 20F consist of a center conductor surrounded by a concentric outer shielding layer made of conductive materials. The center conductor is insulated with the outer shielding layer. The center conductors of the at least two coaxial cables 20E, 20F connect the at least two electrodes 20A, 20B to the microprocessor 30 for transmitting the signal. The outer shielding layers of the at least two coaxial cables 20E, 20F are connected to the reference point with zero potential, e.g., the negative pole of a battery 50. Alternatively, the outer shielding layers are connected to the microprocessor 30 for interference filtering process. However, if the electric environment is very noisy, and the interference becomes too strong to perform a normal operation of this monitoring system, the signal interference from the environment can be removed by special signal processing methods described in FIG. 4B. Further alternatively, two pairs of electrodes can be positioned in parallel outside the IV bottle 11. By differentiation of the signal or electric parameters, the environmental interference will be removed too. Hereby there is no need of grounding in order to avoid the environmental interference since this monitoring system is designed as a portable device.

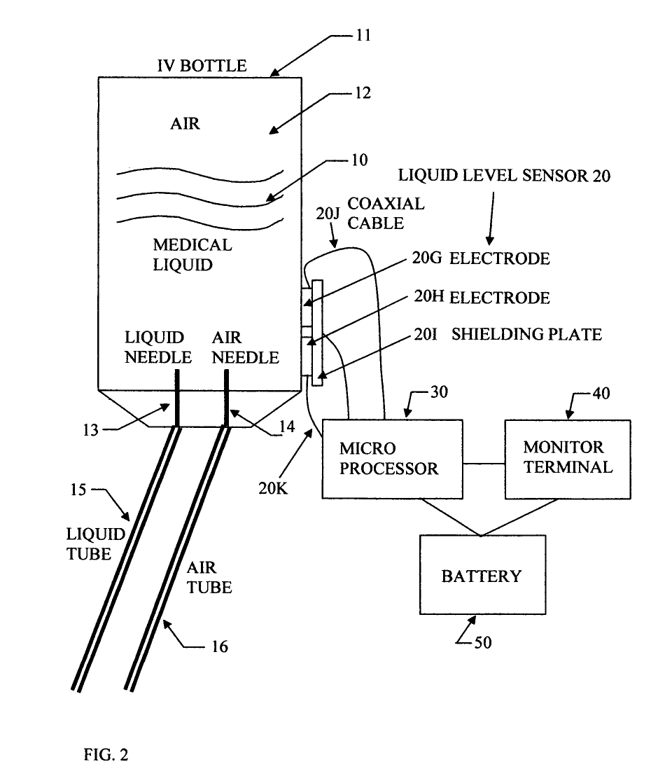

FIG. 2 is the schematic drawing of an alternative embodiment of the present invention. It is similar to the embodiment of FIG. 1, but the liquid level sensor 20 includes at least two electrodes 20G, 20H, which are positioned at one side of the IV bottle 11 in parallel location.

Again, for removing the interference from the environment, at least one shielding plate 20I made of conductive materials is placed on the outer surface of each electrode 20G, 20H. The at least one shielding plate 20I is connected to a reference point with zero potential, e.g., the negative pole of a battery 50. Alternatively, the interference noise in the at least one shielding plate 20I is passed over to the microprocessor 30, and it is then filtered out in signal processing. Meanwhile, at least two coaxial cables 20J, 20K consist of a center conductor surrounded by a concentric outer shielding layer made of conductive materials. The center conductors of the at least two coaxial cables 20J, 20K connect the at least two electrodes 20G, 20H to the microprocessor 30 for transmitting the signal. The outer shielding layers of the at least two coaxial cables 20J, 20K are connected to the reference point with zero potential, e.g., the negative pole of a battery 50. Alternatively, the outer shielding layers are connected to the microprocessor 30 for interference filtering process. However, if the electric environment is too noisy to perform the normal operation of this monitoring system, the signal processing methods described in FIG. 4B can be applied to remove most environmental interference. Further alternatively, two pairs of electrodes can be positioned in parallel outside the IV bottle. By differentiation of the signal or electric parameters, the environmental interference will be removed too.

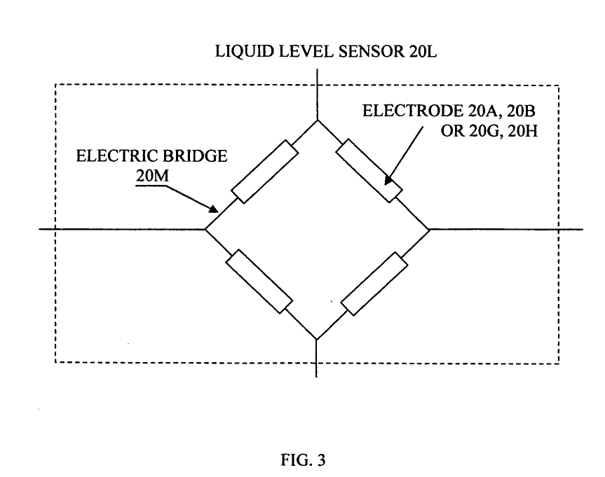

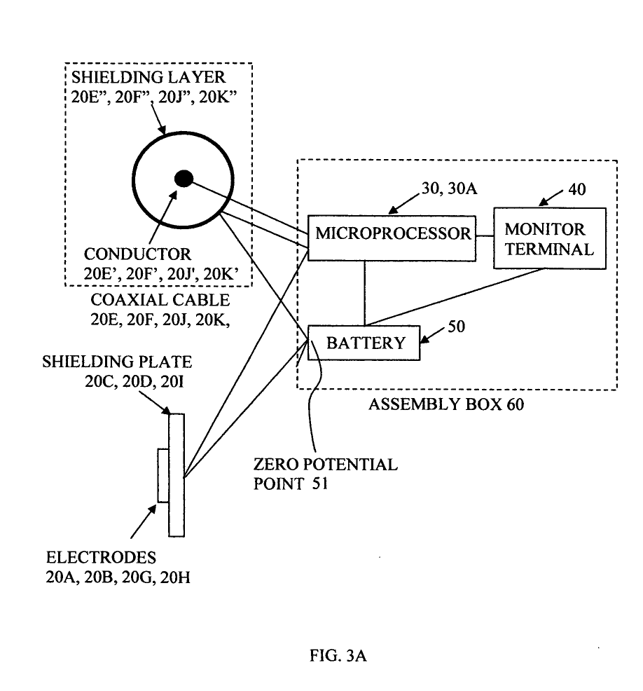

FIG. 3 is a schematic drawing of an exemplary liquid level sensor 20L. The liquid level sensor 20L uses an electric bridge 20M to detect the electric signal of the alternating current. The electric bridge 20M contains the at least two electrodes 20A, 20B in FIG. 1, or 20G, 20H in FIG. 2.

FIG. 3A is a schematic drawing of an exemplary coaxial cable, shielding plate and assembly box for removing the environmental interference. The at least one shielding plate 20C, 20D, 20I is connected to a reference point with zero potential 51, e.g., the negative pole of a battery 50. The at least one shielding plate 20C, 20D, 20I is insulated from the electrodes 20A, 20B, 20G, 20H. Alternatively, the interference signal in the at least one shielding plate 20C, 20D, 20I is passed over to the microprocessor 30, 30A and it is then filtered out in signal processing. Meanwhile, at least two coaxial cables 20E, 20F, 20J, 20K consist of a center conductor 20E′, 20F′, 20J′, 20K′ surrounded by a concentric outer shielding layer 20E″, 20F″, 20J″, 20K″ made of conductive materials. The center conductors 20E′, 20F′, 20J′, 20K′ are insulated from the outer shielding layers 20E″, 20F″, 20J″, 20K″. The center conductors 20E′, 20F′, 20J′, 20K′ of the at least two coaxial cables 20E, 20F, 20J, 20K connect the at least two electrodes 20A, 20B, 20G, 20H to the microprocessor 30, 30A for transmitting the signal. The outer shielding layers 20E″, 20F″, 20J″, 20K″ of the at least two coaxial cables 20E, 20F, 20J, 20K are connected to the reference point with zero potential 51, e.g., the negative pole of a battery 50. Alternatively, the outer shielding layers 20E″, 20F″, 20J″, 20K″ are connected to the microprocessor 30, 30A for interference filtering process. The at least two coaxial cables 20E, 20F, 20J, 20K are in a form of wire, or string, or strip, or twisted-pair, or cable. The center conductor 20E′, 20F′, 20J′, 20K′ is made of solid conductor, e.g., copper in a form of single wire, stranded wires or twist-pair (i.e., two insulated strands of conductive wire twisted around each other). The outer shielding layer 20E″, 20F″, 20J″, 20K″ is made of at least one foil insulation and braided metal, for example, it could be dual shielding (i.e., one layer of foil insulation and one layer of braided metal shielding), or quad shielding (i.e., two layers of foil insulation and two layers of braided metal shielding) if the environmental interference is strong. The coaxial cable 20E, 20F, 20J, 20K has high resistance not only to noise interference, but also to attenuation. The microprocessor 30, 30A, part of the monitor terminal 40 and the battery 50 are contained in an assembly box 60. To shielding the microprocessor 30, 30A and other parts inside the assembly box 60 from the environmental interference, either the assembly box 60 is made of metal or the assembly box 60 is coated with conductive materials. The coated methods include chemical coating, physical coating, mechanical coating, or a simple metal lining etc. Similar to the shielding plate 20C, 20D, 20I, the conductive part of the assembly box 60 is connected to a reference point with zero potential 51, e.g., the negative pole of the battery 50. Alternatively, the environmental noise in the assembly box 60 is passed over to the microprocessor 30, 30A for interference filtering.

FIG. 4 is a block diagram of an exemplary microprocessor 30. The microprocessor 30 acting as a mini computer includes a control means 31 for applying the alternating current to the at least two electrodes 20A, 20B in FIGS. 1 and 20E, 20F in FIG. 2, receiver means 32 for receiving the electric signal of the alternating current, detector means 33 for detecting the electric parameters of the electric signal, A/D converter means 34 for converting the detected analog electric parameters into a group of digital data points, process means 35 for analyzing the group of digital data points by statistics, which then simultaneously generates both the liquid level and liquid flow rate inside the IV bottle 11, as well as transmission means 36 for sending out the liquid level data. Each of above elements may be built together in one chip, or they can stand alone as individual circuit or chip. The applied statistical analysis includes at least one of data filtering, curve fitting and statistical modeling to a group of numerous data points as a function of time, and statistical method to remove electronic noise. The control means 31 includes at least one of an oscillator, an oscillator circuit, a logic circuit etc. The receiver means 32 includes at least one of an input port, an amplifier, a filter etc. The detector means 33 includes at least one of a CN converter (capacitance to voltage converter), a differential circuit, or a voltage meter etc. The A/D converter means 34 includes at least one A/D converter. The process means 35 includes at least one of signal interface, digital register, processor, or logic circuit etc. The transmission means 36 includes at least one of output port, conductive wire or antenna etc.

FIG. 4A is a block diagram of an alternative microprocessor 30A. The microprocessor 30A comprises receiver 32A for receiving the electric signal of the alternating current, detector 33A for detecting the voltage signal from the electric signal, signal interface 34A for storing the voltage signal, A/D converter 34B for converting the voltage signal into a group of digital data points, digital register 34C for storing the group of digital data points, processor 35A for analyzing the group of digital data points as a function of time by statistics and simultaneously obtaining the liquid level data including both liquid level and liquid flow rate of the medical liquid 10 in the IV bottle 11, output port 36A for transmitting the liquid level data. All the functions of each element are controlled by program controller 36B, which is programmed with unique software code for administrating the operation of all above elements. Each of above elements may be built together in one chip, or they can stand alone as individual circuit or chip.

FIG. 4B is a block diagram of three alternative embodiments of the interference filtering means 37, 37A, 37B, which are included in microprocessor 30B, 30C, 30D respectively. The control means 31 in microprocessor 30, 30A of FIGS. 4 and 4A generates the alternating current, which is in various forms including narrow band signal 38, multi-frequency signal 38A or an encode signal 38B. These signals are passed over to the liquid level sensor 20, and then received by microprocessor 30, 30A, 30B, 30C, 30D. For a narrow band signal 38, the interference filtering means 37 includes a narrow band filter 39, which can filter out the signal within this narrow band, and removing all random interference outside the narrow band. For a multi-frequency signal 38A, the interference filtering means 37A includes a Fourier analyzer 39A, which can perform Fourier analysis to pick up the right signal, and remove the noise interference. For an encode signal 38B, the interference filtering means 37B includes a decoder 39B, which can perform decoding to pick up the right signal, and remove the noise interference. The form of signal includes single frequency signal, continuous wave, pulse signal, impulse signal, digital signal, spread spectrum signal and encoded signal etc. The way of encoding includes frequency modulation, angle modulation, phase modulation, pulse modulation, pulse code modulation, FDMA and CDMA modulations etc. All the above interference filtering methods are more effective in digital format.

In addition to the environmental interference, the signal deformation may also reduce the reliability of the monitoring system, e.g., in the case of flexible IV bag, the bag may deform during infusion process and therefore lead to the deformation of the electrical signal and related electrical parameters. However, such signal deformation can be analyzed by the microprocessor 30, and the corrected electric parameters can be picked up by the analysis. Therefore, it would be impossible to obtain high accuracy and high reliability without the microprocessor 30.

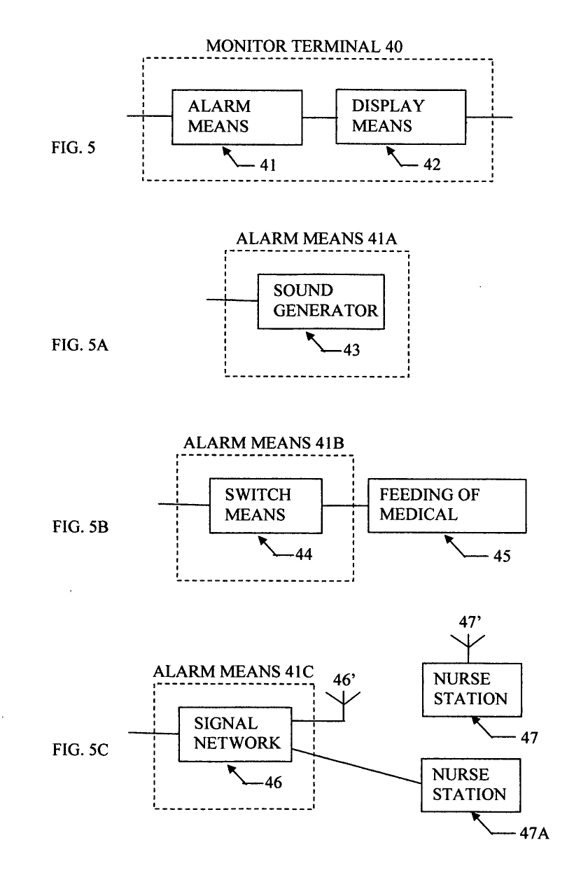

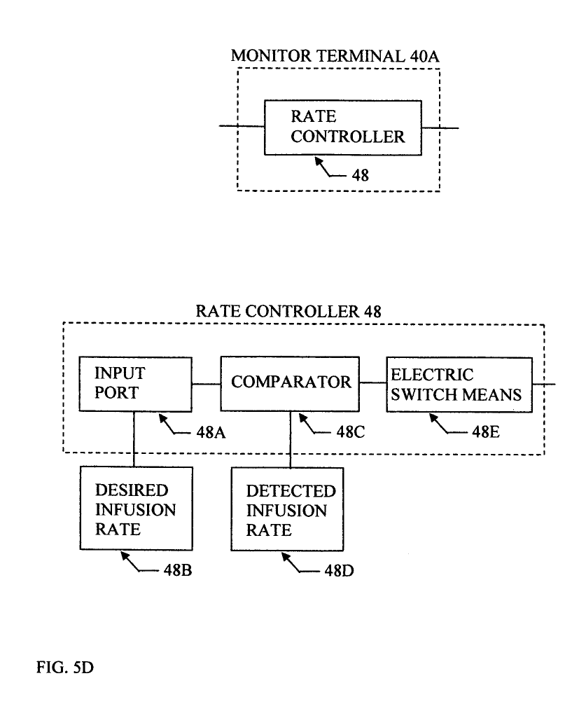

FIG. 5 is a block diagram of an exemplary monitor terminal 40. The monitor terminal 40 includes alarm means 41 for providing an alarm, and display means 42 for displaying the liquid level data in a terminal screen.

FIG. 5A is a block diagram of an exemplary alarm means 41A. The alarm means 41A includes a sound generator 43 for giving a loud sound when the medical liquid level inside the IV bottle 11 in FIGS. 1, 2 drops to the predetermined low level.

FIG. 5B is a block diagram of an alternative alarm means 41B. The alarm means 41B includes a switch means 44 for cutting off the feeding of medical liquid 45 when the medical liquid level inside the IV bottle 11 in FIGS. 1, 2 drops to the predetermined low level.

FIG. 5C is a block diagram of another alternative alarm means 41C. The alarm means 41C including a signal network 46 for sending the liquid level data by the signal network 46 to either a nurse station 47 wirelessly from an antenna 46′ to an antenna 47″, or a nurse station 47A by wire.

FIG. 5D is a block diagram of an alternative monitor terminal 40A. The monitor terminal 40A comprises a rate controller 48 for controlling the infusion rate according to a predetermined rate value. The rate controller 48 includes an input port 48A for inputting the desired infusion rate 48B of the medical liquid 10 inside the IV bottle 11, a comparator 48C for comparing the desired infusion rate 48B and the detected infusion rate 48D, and an electric switch means 48E for adjusting the infusion rate according to the results from the comparator.

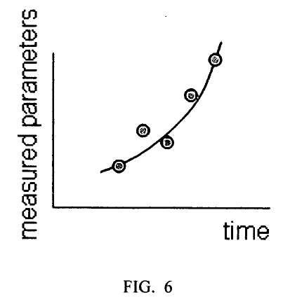

FIG. 6 is a schematic diagram of an exemplary statistical analysis for the present invention. It shows that a group of measured digital data (i.e., measured parameters) is fitted statistically by a smooth curve as a function of time. This statistical analysis requires curve modeling and results in noise filtering. The simplest treatment of such analysis is to calculate simultaneously the average value of the measured parameters and the slope of this curve, which respectively gives the liquid level and liquid flow rate of the medical liquid 10 in the IV bottle 11 for the IV monitoring application. Therefore, the applied statistical analysis of the present invention comprises 2 key factors: (a) measuring a group of digital data points as a function of time; (b) using statistical technique, e.g. curve fitting and statistical modeling etc. The results are derived from the modeled curve. Hereby, the statistical analysis excludes any algebra method, e.g., comparing one point to another point in Cassidy's reference. The statistical analysis for the present invention includes at least one of data filtering, curve fitting and statistical modeling to a group of numerous data points as a function of time, and statistical method to remove electronic noise due to environment.

What is claims is:

1. A portable IV infusion monitoring system, comprising:
(a) a liquid level sensor consisting of two electrodes, an IV bottle comprising at least one of a glass bottle, a plastic bottle, and a plastic bag, said IV bottle containing medical liquid in lower part and air above said medical liquid, said two electrodes being disposed outside said IV bottle for conducting an electric current between said two electrodes, and for measuring interface between said medical liquid and said air above said medical liquid in said IV bottle,
(b) a microprocessor having
control means for controlling said electric current between said two electrodes,
receiver means for receiving an electric signal of said electric current,
detector means for detecting electric parameters of said electric signal, said electric parameters comprising as least one of voltage and impedance,
A/D converter means for converting said electric parameters into a group of digital data points,
process means having a program for analyzing said a group of digital data points by statistical analysis, and obtaining both liquid level and liquid flow rate of said medical liquid simultaneously by said statistical analysis, said statistical analysis including at least one of data filtering, curve fitting and statistical modeling to a group of numerous data points as a function of time, and statistical method to remove electronic noise,
transmission means for sending out liquid level data to a monitor terminal, said liquid level data including said both liquid level and liquid flow rate,
(c) said monitor terminal having
alarm means responsive to said liquid level for giving alarm,
(d) a battery for providing an electric power to said monitoring system, said battery having a negative pole for providing a zero potential reference point to said monitoring system, all parts in said monitoring system being non-grounded, said portable being defined as a function such that said IV infusion monitoring system can operate while moving around with a patient.

2. The monitoring system of claim 1, wherein said two electrodes being positioned at selected one of two sides in opposite direction and one side in parallel location of said IV bottle.

3. The monitoring system of claim 1, wherein said liquid level sensor further comprising at least two coaxial cables for connecting said two electrodes to said microprocessor, and the outer shielding layers of said at least two coaxial cables being connected to at least one of said microprocessor and said zero potential reference point.

4. The monitoring system of claim 1, wherein at least one shielding plate made of conductive materials being positioned at the outer surface of said two electrodes, and being connected to at least one of said microprocessor and said zero potential reference point.

5. The monitoring system of claim 1, wherein said monitoring system comprising an assembly box for containing said battery, said microprocessor, and part of said monitor terminal, and further having shielding means for shielding said microprocessor from environmental interference, said shielding means including selected one of said assembly box being made of metal and said assembly box being coated with conductive materials, said shielding means being connected to selected at least one of said microprocessor and said zero potential reference point.

6. The monitoring system of claim 1, wherein said liquid level sensor further comprising an electric bridge circuit for detecting said electric signal between said two electrodes.

7. The monitoring system of claim 1, wherein said microprocessor comprising
(a) receiver for receiving said electric current,
(b) detector for detecting voltage signal of said electric current,
(c) signal interface for storing said voltage signal,
(d) A/D converter for converting said voltage signal into a group of digital data,
(e) digital register for storing said a group of digital data,
(f) processor for analyzing said a group of digital data by statistics and obtaining said both liquid level and liquid flow rate by said statistics,
(g) output port for transmitting said both liquid level and liquid flow rate,
(h) program controller being programmed with unique software code for administrating the operation of all members from (a) to (g).

8. The monitoring system of claim 1, wherein said control means in said microprocessor further comprising means for controlling said electric current selected from the group including
(a) narrow band signal,
(b) multi frequency signal,
(c) encoded signal, and
said microprocessor further comprising interference filtering means for removing environmental interference, and said interference filtering means comprising a method selected from the group including
(a) narrow band filter for filtering out said narrow band signal,
(b) Fourier analyzer for picking up said multi frequency signal by Fourier analysis,
(c) decoder for decoding said encoded signal.

9. The monitoring system of claim 1, wherein said monitor terminal, comprising at least one device selected from the group consisting of
(a) a sound generator responsive to said liquid level for generating a loud sound when said liquid level inside said IV bottle drops to a predetermined low level.
(b) a switch means responsive to said liquid level for cutting off feeding of said medical liquid within said IV bottle when said liquid level inside said IV bottle drops to said predetermined low level.
(c) a signal network responsive to said liquid level data for transmitting said liquid level data through signal network to a nurse station by a way selected from one of wire transmission and wireless transmission.
(d) display means for displaying said liquid level data.

10. The monitoring system of claim 1, wherein said monitor terminal, further comprising a rate controller including
(a) an input port for inputting desired infusion rate of said medical liquid inside said IV bottle,
(b) a comparator for comparing said desired infusion rate and said liquid flow rate,
(c) an electric switch means for adjusting said liquid flow rate according to results from said comparator.

11. A portable IV infusion monitoring system, comprising:
(a) a liquid level sensor consisting of two electrodes, an IV bottle comprising at least one of a glass bottle, a plastic bottle, and a plastic bag, said IV bottle containing medical liquid in lower part and air above said medical liquid, said two electrodes being disposed outside said IV bottle for conducting an electric current between said two electrodes, and for detecting liquid level of said medical liquid, said liquid level being defined as interface between said medical liquid and said air above said medical liquid in said IV bottle,
(b) a microprocessor having
control means for controlling said electric current between said two electrodes,
receiver means for receiving an electric signal of said electric current,
detector means for detecting electric parameters of said electric signal, said electric parameters comprising as least one of voltage and impedance,
A/D converter means for converting said electric parameters into a group of digital data points,
process means having a program for analyzing said a group of digital data points by statistical analysis, and obtaining both liquid level and liquid flow rate of said medical liquid simultaneously by said statistical analysis, said statistical analysis including at least one of data filtering, curve fitting and statistical modeling to a group of numerous data points as a function of time, and statistical method to remove electronic noise,
transmission means for sending out liquid level data to a monitor terminal, said liquid level data including said both liquid level and liquid flow rate,
(c) said monitor terminal having
alarm means responsive to said liquid level for giving alarm,
(d) a battery for providing an electric power to said monitoring system, said battery having a negative pole for providing a zero potential reference point to said monitoring system, all parts in said monitoring system being non-grounded, said portable being defined as a function such that said IV infusion monitoring system can operate while moving around with a patient.

12. The monitoring system of claim 11, wherein said two electrodes being positioned at selected one of two sides in opposite direction and one side in parallel location of said IV bottle.

13. The monitoring system of claim 11, wherein said liquid level sensor further comprising an electric bridge circuit for detecting said electric signal between said two electrodes.

14. The monitoring system of claim 11, wherein said microprocessor comprising
(a) receiver for receiving said electric current,
(b) detector for detecting voltage signal of said electric current,
(c) signal interface for storing said voltage signal,
(d) A/D converter for converting said voltage signal into a group of digital data,
(e) digital register for storing said a group of digital data,
(f) processor for analyzing said a group of digital data by statistics and obtaining said both liquid level and liquid flow rate by said statistics,
(g) output port for transmitting said both liquid level and liquid flow rate,
(h) program controller being programmed with unique software code for administrating the operation of all members from (a) to (g).

15. The monitoring system of claim 11, wherein said monitor terminal, comprising at least one device selected from the group consisting of
(a) a sound generator responsive to said liquid level for generating a loud sound when said liquid level inside said IV bottle drops to a predetermined low level.
(b) a switch means responsive to said liquid level for cutting off feeding of said medical liquid within said IV bottle when said liquid level inside said IV bottle drops to said predetermined low level.
(c) a signal network responsive to said liquid level data for transmitting said liquid level data through signal network to a nurse station by a way selected from one of wire transmission and wireless transmission.
(d) display means for displaying said liquid level data.
